(19) United States
(12) Patent Application Publication
(10) Pub. No.: US 2020/0096511 A1
(43) Pub. Date: Mar. 26, 2020

(54) SAMPLE DESTRUCTION VALIDATION SYSTEM AND METHODS

(71) Applicant: NANTKWEST, INC., Culver City, CA (US)

(72) Inventor: Nicholas J. Witchey, Culver City, CA (US)

(21) Appl. No.: 16/576,415

(22) Filed: Sep. 19, 2019

Related U.S. Application Data
(60) Provisional application No. 62/733,817, filed on Sep. 20, 2018.

(51) Int. Cl. G01N 33/573

(52) U.S. Cl. CPC G01N 33/573; G01N 2469/00

(57) ABSTRACT

Embodiments of the present invention allow for verifying the destruction of cells by another party. The destruction of cells can be verified by generating a value of an indicator (e.g., a bar code), where the value depends on some feature of the destruction. The party destroying the cells provides the value of the indicator as evidence that the cells have been destroyed. Embodiments may include a method of verifying the destruction of a cell. The method may include running a medium including a dead cell through an assay using a system. Running the medium through the assay may generate a value of an indicator to signify that the assay has been run. The value may identify the system, the cell, or the destruction mode of the cell. The method may further include receiving a verification code to acknowledge that the value is valid.

### PRIORITY CLAIM

This application claims priority to our copending U.S. Provisional Patent Application with the Ser. No. 62/733,817, which was filed Sep. 20, 2018, which is incorporated by reference herein.

### BACKGROUND

In some instances, the owner of a cell line may direct the user of a cell line to destroy samples containing cells of the cell line. Conventional methods of demonstrating destruction of the cells involve taking a photo of a sample vial in a biohazard waste bag. Such methods do not provide actual verification that the cells have been destroyed. Hence, a method to verify the destruction of the cells at some level is needed. These and other needs and improvements are addressed in this disclosure.

### BRIEF SUMMARY

Embodiments of the present invention allow for verifying the destruction of cells by another party. The destruction of cells can be verified by generating a value of an indicator (e.g., a bar code, QR code, color change, etc.), where the value depends on some feature or characteristics resulting from the cell destruction. The party destroying the cells provides the value of the indicator as evidence that the cells have been destroyed. Embodiments include methods and systems related to verifying destruction of cells.

Embodiments may include a method of verifying the destruction of a cell. The method may include running a medium including a dead cell through an assay using a system. Running the medium through the assay may cause generation of a value of an indicator to signify that the assay has been run. The value may identify the system, the cell, or the destruction mode of the cell. The method may also include transmitting the value to a processor. The method may further include receiving a verification code to acknowledge that the value is valid.

Embodiments may include a method of verifying the destruction of a cell. The method may include encoding a system with an indicator having a value. The value of the indicator may be generated after a medium including a dead cell is run through an assay using the system. The value may identify the system, the cell, or the destruction mode of the cell. The method may also include receiving the value. The method may further include comparing the value to a reference value. Additionally, the method may include transmitting a verification code based on the comparison of the value to the reference value.

Embodiments may include a kit for verifying the destruction of a cell. The kit may include an agent configured to destroy the cell. The kit may also include a system for running an assay to detect a component in either the agent or a medium comprising a destroyed cell and the agent. The system may be encoded with an indicator having a value. The value of the indicator may be generated after a medium is run through the assay using the system.

A better understanding of the nature and advantages of embodiments of the present invention may be gained with reference to the following detailed description and the accompanying drawings.

### BRIEF DESCRIPTION OF THE DRAWINGS

FIG. 1 illustrates a process for tracking the destruction of cell according to embodiments of the present invention.

FIG. 2 shows a lateral flow assay architecture according to embodiments of the present invention.

FIG. 3 illustrates components of the indicator value according to embodiments of the present invention.

FIG. 4 is a method of verifying destruction of a cell according to embodiments of the present invention.

FIG. 5 is a method of verifying destruction of a cell according to embodiments of the present invention.

FIG. 6 is a system for analyzing a cell medium according to embodiments of the present invention.

FIG. 7 shows a computer system according to embodiments of the present invention.

### DETAILED DESCRIPTION

Conventional methods of verifying destruction of cells do not include a way to show that cells are actually destroyed or even mixed with an agent to kill the cells. Typical methods to verify destruction include providing a photo of a sample vial in a biohazard waste bag. These methods do not provide strong verification that the cells were destroyed. The vials could be simply taken out of the biohazard waste bag after the photo. Additionally, vials that do not contain the cells could have been used in the photo instead of vials with the specific cells.

Embodiments of the present invention include verification of the destruction of cells by providing a value of an indicator that is associated with the destruction of the cells. The value of the indicator may identify an agent used to destroy the cells, the type of cell, or the system used for running the assay. The value of the indicator may not be known to the party tasked with destroying the cells prior to destroying the cells. As a result, providing the value of the indicator can verify destruction of the cells.

### I. Process Flow

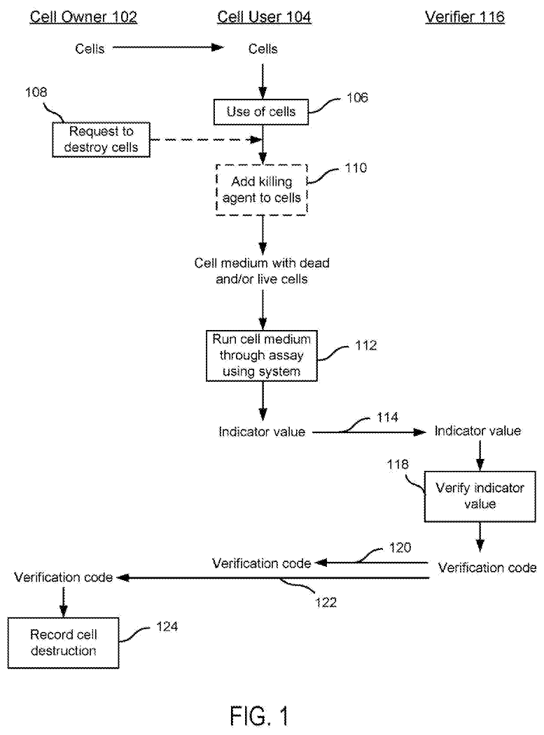

FIG. 1 shows an embodiment of a process involving tracking the destruction of cells. A general overview of the process is presented. Specifics regarding components or steps of the process are described below.

Cell owner 102 may own cells or have the ability to license out the cells. The cells may be sent to cell user 106. The sending of the cells may be through an intermediary. Cell user 104 may use the cells (block 106). The cells may be used in a study or experiment.

Cell owner 102 may request cell user 104 destroy the cells (block 108) under their rights within a license agreement entered by cell user 104. The request to destroy the cells may be included in the license under which cell user 104 is allowed to use the cells. In some embodiments, cell owner 102 may make a specific request to cell user 104 after cell user 104 is already in possession of the cells. It is specifically contemplated that such licensed can include requirements that cell user 104 would be required to use the disclosed technologies for cell destruction. Further, the licenses can be embodied as a “smart contract” implemented on a distributed ledger technology. For example, the license and its terms can be implemented via the Ethereum blockchain, which can record the contract as well as its terms and conditions.

Cell user 104 may then add an agent to the cells (block 110). In some embodiments, no agent is used, but instead the cells may undergo apoptosis or natural death. As a result of the agent, a cell medium may be formed. The cell medium may include a dead cell (or cells), cell fragments, lysate, and/or a live cell (or cells). As such, the cell medium could include many different types of cell fragment to which the disclose assay could be sensitive. Example cell fragments include mitochondria, DNA, RNA, membranes, organelles, or other portions of the cell.

The cell medium may be run through an assay using a system (block 112). An indicator value may be generated by the system after the cell medium is run through the assay. For example, specific organic lysates from the dead cells can interact with chemicals or compounds of an indicator to cause generation of the indicator value or reveal the indicator value.

The indicator value is sent, as denoted by arrow 114, to verifier 116. Verifier 116 verifies the indicator value (block 118). Verifying the indicator value produces a verification code.

The verification code may be sent to cell user 104, as indicated by arrow 120. In some embodiments, cell user 104 is not sent the verification code. In some embodiments, the verification code is sent to cell owner 102, as indicated by arrow 122. Sending the verification code may be through electronic communication, including cellular signals and Internet communication.

Cell owner 102 may then record cell destruction (block 124). Recording the cell destruction may be through updating a database or updating a blockchain.

### II. Cells

Cells that may be tracked for destruction may include cell lines and microorganisms (e.g., bacteria). The cells may be cells that are stored and distributed by an organization (e.g., American Type Culture Collection [ATCC]).

The cells may include cells from at least one of the following tissues: adipose, aorta, artery, blood, cord blood, bone, bone marrow, brain, breast, cervix, colon, eye, heart, small intestine, large intestine, kidney, liver, lung, lymph node, muscle, ovary, pancreas, pituitary, prostate, retina, skin, spleen, or vein. The cells may include stem cells and/or cells of the immune system such as dendritic cells, NK cells, T cells, B cells, etc., all of which may be genetically modified to express on or more recombinant proteins (e.g., a CAR, a cytokine, a cytokine analog such as N-803 or TxM type construct, cancer- and patient specific neoantigens as single peptides or polytopes, etc.). Further, the cells could other types of cellular organism. For example, the disclosed techniques could also be leveraged for yeast, bacteria, protozoa, or other organisms.

The cells may include NK-92 cells. NK-92 cells from a cell line that is similar to natural killer (NK) cells in the blood. NK-92 cells may be used to attack tumor cells. Embodiments may include other NK cells. The cells may include T cells (i.e., T lymphocytes) or B cells. The cells may be tagged, including with a chemical or genomic moiety.

In some embodiments, methods may be used to track the destruction of viruses instead of cells.

### III. Agents

Agents used to kill or destroy the cell may include a lysing agent, ultraviolet radiation, an enzyme, an antiseptic, a cytotoxin, a virus, a bacteria, a fungus, heat, cold, a cytolysis agent, an acid, a base, a white blood cell, centrifugal force, ultrasound, and/or high pressure.

A lysing agent may include a virus, an enzyme, or an antibiotic. An enzyme may include a protease and/or lysozyme.

The agent may include perforin, which refers to a protein that binds to a cell and forms pores in the cell.

The agent may include ultraviolet radiation. Ultraviolet radiation may electromagnetic radiation with a wavelength from 10 nm to 400 nm.

Antiseptics include alcohols (e.g., ethanol, isopropyl alcohol), chlorohexidine gluconate, hydrogen peroxide, iodine, octenide, dihydrochloride, polyhexanide, sodium hypochloride, or bleach.

A virus, a bacteria, or a fungus may be used to kill the cell. The virus may lyse the cell or may leave the cell with a portion of the cell membrane. A bacteria or fungus may kill the cell through any suitable means, including destroying the cell membrane.

The agent may be result of changing the temperature. Heat may be added, including heat greater than or equal to 80° C., 90° C., or 100° C. Cells may be subject to cold so that cell membranes freeze and are destroyed or by some other mechanism. Temperatures may be lower than or equal to 0° C., −50° C., −100° C., −150° C., −200° C., or −250° C. Temperatures may also be between any two temperatures disclosed.

A cytolysis agent may include a fluid with a high concentration of water. As a result of osmotic effects, water moves into the cell and eventually causes the cell to burst.

The agent may affect the pH. An acid or base may attack and destroy the cell wall or cell components, which lead to the destruction of the cell. In some instances, the acid or base may prevent certain biochemical pathways from proceeding, which kills the cell. The acid may have a pH from 1 to 7. The base may have a pH from 7 to 14. The acid may include a Bronsted acid or a Lewis acid. Similarly, the base may include Bronsted base or a Lewis base.

A white blood cell or other immune system related-cell may be used to kill the cell. White blood cells may include leukocytes, granulocytes, agranulocytes, myeloid cells, lymphoid cells, neutrophils, eosinophils, basophils, lymphocytes, and monocytes. Lymphocytes may include B cells, T cells, or NK cells.

High pressure may be applied to destroy cells. Pressures may include a pressure from 100 MPa to 500 MPa, from 500 MPa to 1,000 MPa, or greater than or equal to 1,000 MPa. Alternatively, or additionally, ultrasound (sonication) may be employed to disrupt the cells.

In some embodiments, the agent is a fluid (e.g., a gas or liquid), and the agent includes a compound that does not directly or indirectly cause the death of cell. The compound may be a tag, for example, a fluorescent label, which identifies the specific agent used. In some embodiments, the agent may include a reactant compound, which may react later with a reactant in an assay.

In some embodiments, no agent is used, and the cell dies from apoptosis instead.

### IV. Medium and Markers for Cell Death

The cell medium may include the agent and products or byproducts of the reaction with the agent and/or the dead cells. Dead cells include necrotic cells or apoptotic cells. Necrotic cells typically display organelle swelling, increased permeability with loss of cell membrane integrity, and the release of intracellular contents. Apoptotic cells typically show cell shrinkage, nuclear fragmentation, and activation of a family of cysteine-containing, aspartate-directed proteases called caspases. Apoptotic cells eventually become necrotic cells. Because not all cells may be killed or destroyed, the medium may include live cells. If a cell is lysed, the cell medium may include fragments of the cell membrane as well as cell-free organelles and other internal components of the cell, and the cell medium may be called the lysate.

The medium may include compounds or elements in a particular pattern or relative abundance. The particular pattern or relative abundance may be different from the pattern or relative abundance in the agent before contacting the agent with the cells and different from the pattern or relative abundance in the cells and accompany liquid before contacting with the agent. The particular pattern or relative abundance may be a signature or a fingerprint to identify that the agent has contacted a live cell or cells.

Numerous cell death markers can be measured to assess the extent of cell destruction. Cell death can be evaluated using measurement of mitochondrial dehydrogenase activities (Fanning et al. Gynecol. Oncol. 39: 119-122 (1990)), cellular respiration (Schenellmann, Vitro Toxicity Indicators. Academic Press; San Diego: 128-139 (1994)), and mitochondrial membrane potential using the flurometric dyes 5, 5′6,6′-tetrachloro-1,1′3,3′-tetraethylbenzimidazocarbocyanine iodide (JC-1) and tetramethylrhodamine methylester, both available from Invitrogen (Reers et al., Methods Enzymol. 1995; 260:406-417 (1995)); Lemasters et al., J. Bioenerg Biomembr. 31:305-319 (1999)); DNA damage (Sorger and Germinario, Anal Biochem. 131:254-256 (1983)); Shen et al. Toxicol Appl Pharmacol. 111:242-254, (1991); Yan et al., Anal Biochem. 286:138-148 (2000)), and cytosolic free Ca2+ levels (Ogden et al., Pflugers Arch. 429:587-591 (1995)) and membrane integrity (Cummings et al., Curr. Protoc. Pharmacol. September 1, doi:10.1002/0471141755.ph1208s25, 2004).

Products or byproducts in the medium after the cells are treated with cell destruction agents may include perforin or granzymes. The granzymes are cell death-inducing enzymes, stored in the cytotoxic granules of cytotoxic T lymphocytes and natural killer cells, which are released during granule exocytosis when a specific virus-infected or transformed target cell is marked for destruction. Granzymes enter the target cell cytosol through plasma membrane pores formed by perforin. Granzymes and perforins can be readily determined using methods well known in the art, for example, ELISA. One exemplary method for measuring granzymes is disclosed in Bade et al. Eur. J. Immunol. 2005 September 35 (9): 2608-16. In other aspects, nucleic acids from the cell may be detected, and especially suitable nucleic acids include genomic DNA and/or mRNA (especially recombinant DNA and/or RNA), ribosomal RNA, mitochondrial DNA (which may be derived from native mitochondria or mitochondria that were previously introduced into the cell), etc.

In some cases, cell death is evaluated by detecting caspases. Caspases are key effectors of apoptotic cell death. Upon activation, caspases are cleaved and the cleaved form (the active form) then cleaves cellular proteins to dismantle the dying cells. The caspases in the cell lysates can be detected using agents that can specifically react with the active form of the caspase or its substrate. For example, the assay can be based on spectrophotometric detection of the chromophore p-nitroaniline (p-NA) after cleavage from the labeled DEVD-pNA, a substrate for caspase 3. Methods for detecting caspase in cell lysate to detect cell death are well known and reagents are commercially available. For example, methods may include using the caspase 3 assay kit from Abcam. See URL www.abcam.com/kits/role-of-caspases-in-apoptosis.

In some cases, cell death is evaluated by assessing release of the cytosolic enzyme lactate dehydrogenase (LDH). The presence of this protein in the extracellular milieu is a marker for loss of cell viability, loss of membrane integrity, and the presence of necrosis. LDH activity can be measured by, for example, incubating the medium with pyruvic acid (the LDH substrate), followed by measuring the signal using 360 nm excitation and 460 nm emission. An exemplary method for assessing LDH is disclosed in (Cummings et al., Curr. Protoc. Pharmacol. September 1, doi:10.1002/0471141755.ph1208s25, 2004).

In some cases, cell death is evaluated by assessing membrane integrity. In some embodiments, plasma membrane permeability is assessed using propidium iodide or lactate dehydrogenase. In some embodiments, the cell death is evaluated by assessing lysosomal membrane integrity using neutral red (Monks et al. 1988, Mertens et al., 1995)

In addition, apoptotic cells can be detected by detecting externalization of phosphatidylserine, e.g., by fluorescently-labeled Annexin V and necrotic cells can be detected by measuring the permeability of the plasma membrane to a normally impermeable fluorescent dye, such as the DNA-binding dye, propidium iodide (PI).

Additional assays for assessing cell destruction may also include the TUNEL assay, which detects the DNA strand breaks associated with cell death. In one embodiment, the cells may be seeded onto slides and stained to label DNA strand breaks with fluorescein-dUTP. One exemplary method is disclosed in https://jeccr.biomedcentral.com/articles/10.1186/s13046-017-0495-3.

Other markers that may be used to track cell death may include cytokines, chemokines, DNA, RNA, or tubulin.

### V. Assay

The assay may include any assay suitable for analyzing the cell medium. One particular assay may be a lateral flow test, run on a lateral flow test substrate. A well-known example of the lateral flow test is an over-the-counter pregnancy test. A lateral flow test may be an inexpensive and easy manufactured assay for verifying destruction of cells.

### Lateral Flow Test

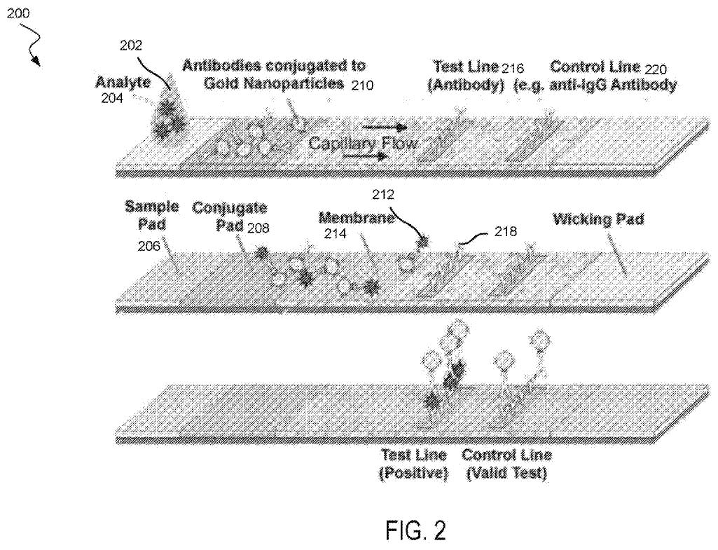

As shown in FIG. 2, the assay may include a lateral flow test 200. A lateral flow test may be used to detect the presence or absence of an analyte using a paper-based device. The analytes may be a specific compound or combination of compounds expected in a cell medium after the agent is applied. Sample fluid 202 with analyte 204 is placed on sample pad 206.

Sample fluid 202 is driven to conjugate pad 208. Conjugate pad 208 initially contains antibodies 210 conjugated with a tag (e.g., gold, latex, fluorophore). In FIG. 2, antibodies 210 are conjugated with gold nanoparticles. Analyte 204 binds with antibody 210 through a chemical reaction.

The bound analyte and antibody conjugated tag 212 is driven by capillary action across membrane 214. The analyte then reaches a test line 216 with immobilized antibodies 218. The analyte binds to the immobilized antibodies. As more analytes are captured by the immobilized antibodies, the test line becomes visible (e.g., by changing color) because of the accumulation of conjugated tags at the test line.

Antibodies that are not bound to the analyte are then captured by an antibody at control line 220. Control line 220 verifies that the reaction conditions were sufficient to move an antibody to the control line. The control line becomes visible with the accumulation of conjugated tags.

Analytes that may be tested may include any compound or combination of compounds in the cell medium. Analytes may include any marker for cell death described herein. Multiple analytes may be tested using the same lateral flow test substrate. Different test lines can be configured to detect different analytes.

The test line and the control line may be patterned to convey information beyond a binary yes/no detection result. The test line and the control line may be patterned into a bar code or other indicator to provide other information. The bar code or other indicator is described in more detail below.

### Other Assays

The assay may include a bioassay, a ligand binding assay, an immunoassay, an enzyme assay, a light detection system, a radioisotope assay, a sequencing assay, a nucleic acid hybridization assay, a polymerase chain reaction assay, a photometry assay, a transmittance assay, a turbidimetry assay, a nephelometry assay, a reflectometry assay, a viscoelastic measurement, a counting assay, an imaging assay, a cytometry assay, or an electrical detection. These assays may operate in a similar manner. The assay may generate a result when a medium with dead cells is run through the assay. The result may be different from when either the agent itself is run through the assay or when the live cells and accompanying liquid are run through the assay.

The agent may include a first reactant and the assay may include a second reactant. The first reactant and the second reactant may react to form a product or products. The resulting product or products may be readily visible. For example, the resulting product may be a precipitate, having a readily visible appearance or color. In some embodiments, the resulting product or products may be readily detectable. For example, the product or product may increase the electrical conductivity of the cells and accompanying liquid. The electrical conductivity can be measured using a voltmeter or a current meter. In some embodiments, the resulting product or products may change the pH of the cell medium, which can be detected with a pH meter or pH paper.

In one embodiment, the assay is a flow cytometry assay. Necrotic cells or apoptotic cells are stained with a fluorescently-labeled agent that recognizes a cell death marker as described above, for example fluorescently labeled PI and/or Annexin V. The stained cells are washed and analyzed on a flow cytometer. The fluorescent signals are correlated with cell death. An exemplary assay is disclosed in www.ncbi.nlm.nih.gov/pmc/articles/PMC3874588/# R26.

In some embodiments, the assay is a plate-based assay. The cell lysate from medium comprising dead cells are coated on a plate. Reagents that are labeled with detectable label that can recognize one of the cell death markers as describe above are incubated with the lysate coated on the plate. The signal from the binding of the labeled reagent to the marker are detected and such signal is positively correlated with cell death.

In some embodiments, the assay is an imaging assay that is used to assess nuclear morphology on cells treated with a cell-permeable nucleic acid stains, such as 4′,6′-diamidino-2-phenylindole (DAPI) and Hoechst 33342. DAPI and Hoechst are two dyes that only stain nucleic acids in cells in which membrane have been permeated. In some embodiments, high content imaging system can be used to automate and quantify changes in nuclear morphology, which correspond to cell death.

### VI. Indicator

Running the assay with the agent and the cell medium may reveal an indicator, which provides information on the assay that has been run. In some embodiments, the indicator may be generated directly through running of the assay. For example, running a lateral flow test may reveal the indicator in the test lines and control lines on the lateral flow test substrate. In other embodiments, the indicator may be revealed as a subsequent step after the assay is run. For example, an assay may detect the agent and the cells through a change in the conductivity. Once a certain threshold conductivity is met, a device may then display the value of the indicator.

The indicator may be a bar code, a QR code, an image, an alphanumeric string, a character string, a number, a line, or a color. For example, the indicator may be a line, similar to the lines used in lateral flow tests including pregnancy tests. The indicator may authenticate that the assay has been run because the value of the indicator is unknown to the entity destroying the cell until the assay has been run. The indicator value and the way it is determined may be hidden or encrypted so that the user of the cell does not know what the indicator value should be until after the assay is run. The image may be a logo or some readily identified image (e.g., a dog, a car, a flower, etc.). The QR code may include a link to a web page or may be a value. The number may be a phone number, which may need to be dialed to verify the assay has been run. The character string may include a URL or a username or password. The value of the indicator (e.g., the URL, the identity of the image, etc.) can then be sent to a verification server in order to verify that the assay has been run and/or that a cell has been destroyed.

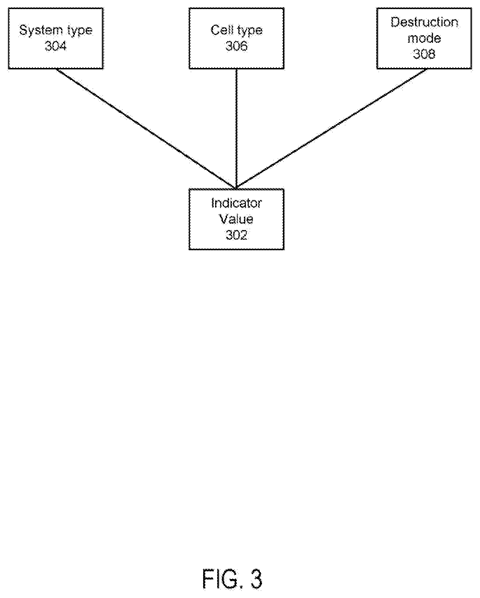

The value of the indicator may provide additional information regarding the cells, including identifying the system, the cell, the destruction mode, a vial, or the destruction efficiency. FIG. 3 shows different portions of indicator value 302. Indicator value 302 may include a portion involving system type 304. System type 304 may be the model number, lot number, or serial number of the system used for the assay. In other words, system type 304 may identify a unique system used or perhaps a category of systems used. For example, system type 304 may be “Unit 125” of 300 units or “Model A” line, which itself includes 300 units. In some embodiments, indicator value 302 may identify the type of assay being run (e.g., lateral flow test).

Indicator value 302 may include a portion involving cell type 306. Cell type 306 may be a type of cell (e.g., NK-92), a cell line, or a specific lot of cells. Indicator value 302 may include information on the vial of the cell (e.g., serial number or lot number of vial or cells in the vial). One should appreciate that the indicator value can take on many different forms that could be designed to be unique to the cell users. Such an indicator value could encode one or more of the following features: a cell user identifier, a cell vial identifier, a cell line identifier, a verification code, a cryptographic key, or other features. Such an approach is considered advantageous because it provides for determining, with specificity, the disposition of specific cells as well as the cell destruction actions taken by the cell user.

Indicator value 302 may also include a portion involving destruction mode 308. Destruction mode 308 of the cell may include identifying the agent used. Destruction mode 308 may also include information on the destruction efficiency. Destruction efficiency may include a numerical value of the efficiency. For example, destruction efficiency may include that a certain percentage or proportion of cells have been destroyed. In some examples, destruction efficiency may include a relative measure of efficiency. For example, “high” or “low” amount of cells destroyed.

In some embodiments, indicator value 302 may include information on the user of the cell. The user of the cell may be identified by name or a number.

### VII. Example Method

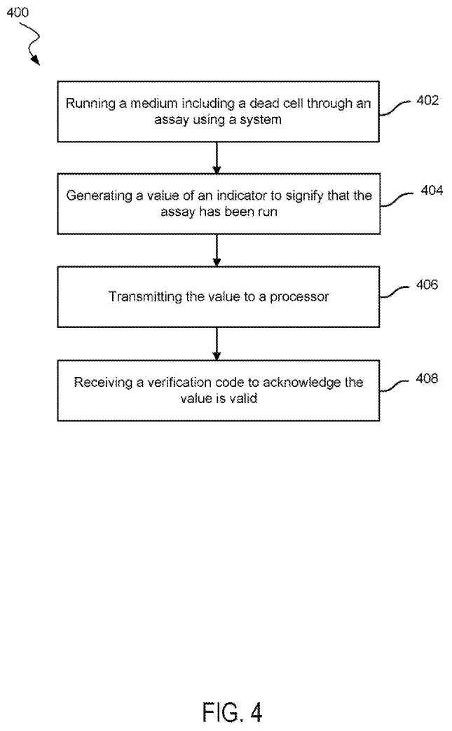

FIG. 4 shows a method 400 of verifying the destruction of a cell. In some embodiments, the method may include destroying the cell using an agent to produce a medium including at least a fragment of the dead cell. The agent may include any agent described herein. The cell may include any cell described herein.

At block 402, method 400 may include running a medium including at least a fragment of a dead cell through an assay using a system. The medium may include the entirety of the dead cell. The assay may be a lateral flow test. The system may include a lateral flow test substrate. The assay may be any assay described herein.

At block 404, running the medium through the assay may generate a value of an indicator to signify that the assay has been run. The indicator may be any indicator described herein. In some embodiments, the indicator may be a light. The value may be the color of the light or the intensity of the light. In embodiments, the indicator may be an electrical or magnetic characteristic. For example, the electrical or magnetic characteristic may be current, resistance, voltage, or a magnetic flux. The value of the indicator may include the magnitude of a characteristic.

The value of the indicator may be generated as the result of detecting a product in the medium. The product may be produced by reacting a first reactant in the agent with a second reactant used in the assay. For example, the product may be a precipitate formed with a certain color, and the color of the medium after running the assay may be the value of the indicator. In some embodiments, the indicator value is only revealed for specific cells. This can be achieved through by tagging the cells with reactants that only interact reactants in the assay.

The value of the indicator may be generated by becoming visible after running the medium through the assay. For example, if the assay is a lateral flow test, the indicator may include a test line, bar code, or QR code that becomes visible when an antibody immobilized on the substrate captures an antigen by reacting with the antigen.

The value may identify the system, the cell, or the destruction mode of the cell. The value may identify the system. The system may be labeled with an identifier (e.g., model number or serial number). The value may depend on the identifier.

The value may identify the cell. The cell may be a type of cell, and the value may identify the type of cell. The value of the indicator may include a portion associated with the type of cell. The value of the indicator may depend on a proportion or an amount of a compound in the medium. For example, certain properties (e.g., pH, light transmittance, color, electrical characteristics) may depend on the amount of the compound. The compound may include perforin, granzymes, or a component specific to cell death. In other words, the compound may be an indicator of death because the compound may increase in concentration with the death of a cell. The value of the indicator may be generated when the proportion or the amount of the compound exceeds a threshold.

The cell may be tagged with a chemical or genomic moiety. The value may depend on the chemical or genomic moiety. For example, the cell may be tagged with a specific chemical moiety. The tag may be a fluorescent label. The cells may be tagged with exogenous mitochondria, plasmids, or nanoparticles. The assay used may be an assay to detect the specific chemical moiety. The value of the indicator generated identifies the specific chemical moiety. Different cells may be tagged with different chemical moieties, which would generate a different value of the indicator.

The value may identify the destruction mode of the cell. The presence of the agent, or a concentration of the agent above a threshold, may cause the value of the indicator to be generated. For example, a medium with the agent having a concentration above a certain amount may have a certain pH level, which may trigger a reading on a pH meter or a certain color using pH paper. The medium may include a plurality of dead cells and a plurality of live cells. The value of the indicator may depend on a proportion of dead cells and live cells.

The method may also include transmitting the value to a processor. Transmitting the value may include sending an image of the value. The processor may then send a verification code to the cell owner (and/or their systems) to acknowledge that the value is valid.

The method may further include receiving the verification code. In some embodiments, the verification codes may consist of a standard cryptographic hash using a private key known by the cell owner (and/or their systems) and the processor.

In some embodiments, the verification code may include a blockchain portion. The blockchain ledger may be an “open” or “closed” distributed ledger, where all cell depositories are nodes therein. Distributed ledgers other than blockchain may also be employed, including hashgraphs, directed acyclic graphs (e.g., IOTA), etc. Blockchain tracking and validation methods may be related to methods descried in US 2015/0332283 and US 2018/0082043, the contents of which are incorporated herein by reference for all purposes. The value or verification code may be related to sample tracking methods and systems described in US 2018/0082043, the contents of which are incorporated herein by reference for all purposes. Further, the cell destruction information can be encoded within a “smart contract”, possibly as recorded on the Ethereum blockchain. In some case, recording of such an event can indicate closure with respect to a license.

In some embodiments, each cell destruction verification block in a verification blockchain may include data regarding a particular cell and its destruction (or a plurality of cells and their destruction). The data regarding the particular cell may include, merely by way of example, cell identifying information and/or any other known characteristics of the cell, including images and any other information obtainable by a transducer subsystem. The data regarding the cell's destruction may include, merely by way of example, any identifying information about the destruction process including date, time, location, substances, equipment identification, methodology and destruction process characteristics, personnel involved, etc. Data regarding the cell's destruction may later be analyzed with or without respect to the cell identifying information to manually or automatically verify that a cell was destroyed, or at least manually or automatically determine a probability that the cell was destroyed.

In some embodiments, each cell destruction verification block in a verification blockchain may also include identifying or characteristic information regarding each system and/or person responsible for requesting, conducting, and verifying the destruction of a cell or cells. Merely by way of example, model information, serial numbers, media access control (MAC) addresses, network identifying information such as Internet Protocols (IPs), personnel identification information (public and/or proprietary), etc. In these embodiments, systems and/or individuals responsible for a successful or unsuccessful request, execution, and verification of a destruction order may later be quickly identified, allowing equipment and personnel functioning out of specification, in accordance with incorrect instructions, and/or not in accordance with correct instructions to be quickly identified.

### VIII. Verification Method

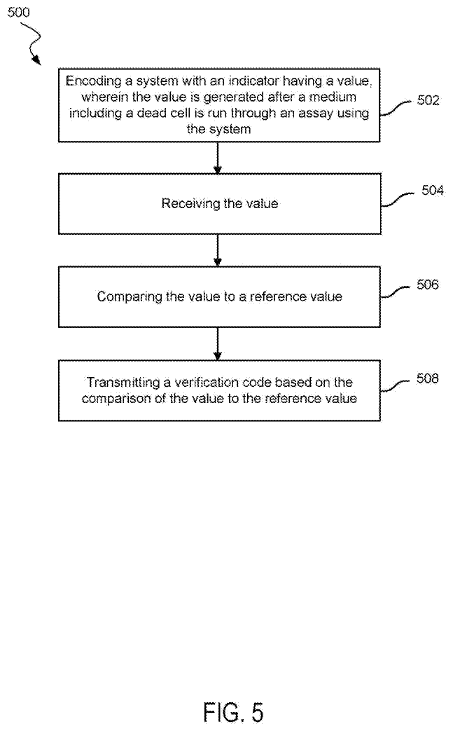

As shown in FIG. 5, embodiments may include a method 500 of verifying the destruction of a cell. At block 502, method 500 may include encoding a system with an indicator having a value. The system may include a lateral flow test substrate or any system related to any assay described herein. Encoding the system with the indicator may include printing the indicator onto the lateral flow test substrate. The system may include any system to run an assay described herein.

The value of the indicator may be generated after a medium including at least a fragment of a dead cell is run through an assay using the system. The medium may include the entirety of the dead cell. The value may be any value described herein.

At block 504, method 500 may also include receiving the value. The value may be received through cellular, wireless, or Internet communication. The value may be transmitted using a software program or an app on a computer, smartphone, tablet, smart watch, or other suitable device. For example, a cell user can use a smartphone, possibly via a dedicated app, to capture a digital image of the indicator value; reading a bar code or recognizing a revealed image. Techniques by which a smartphone could recognize information in images can be found in U.S. Pat. Nos. 6,711,293; 7,016,532; and 7,477,780.

At block 506, method 500 may further include comparing the value to a reference value. The reference value may include a portion that identifies the system, the cell or the destruction mode of the cell. The reference value may be one of a plurality of reference values. The reference value may include a first portion with a type of system and of a plurality of types of systems. The reference value may include a second portion associate with a type of cell of a plurality of types of cells.

The method may further include for each type of system of the plurality of types of systems, assigning a first value uniquely identifying the type of system. For each type of cell of the plurality of types of cells, the method may include assigning a second value uniquely identifying the type of cell. Each reference value may be determined by combining a first value of the plurality of first values with a second value of the plurality of second values.

The reference value may also include a third portion involving the destruction mode or any identifier described herein. The reference value may be determined by combining the first value, second value, third value, or other values. The reference value and the method of determining the reference value may not be known to the entity directing the destruction of the cell.

Method 500 may also include receiving data including an identity of the system and an identity of the cell. The reference value may be generated using the data. For example, a user of the cell may input in their system identity and cell identity using a computer, smartphone, tablet, or other suitable device. A processor, including one on the computer, smartphone, tablet, or a remote verification server, may then determine the reference value based on those inputs.

At block 508, method 500 may include transmitting a verification code based on the comparison of the value to the reference value. The verification code may be transmitted when the value is equal to the reference value. The verification code may simply be a message confirming that the value of the indicator is verified.

In some embodiments, comparing the value to a reference value may be done at or near the location of running the assay. For example, a mobile phone or tablet application may take a photo of the value of the indicator. The value of the indicator may be further processed with image recognition techniques. The application may recognize the indicator (e.g., using Scale-Invariant Feature Transform disclosed U.S. Pat. No. 6,711,293, edge detection, etc.) as being related to at least one of the user, the cell, the vial, the system, or the destruction mode. The application may then transmit the verification code to the user by displaying a code on the screen. The application may then send the validation information to a remote verification server and/or may update the blockchain ledger as discussed above.

In some embodiments, if the value does not match the reference value, the verification code may be a message that the value of the indicator is not verified or directing the user of the cell to run another assay.

In some embodiments, the verification code may be transmitted to the entity directing the destruction of the cell (e.g., user of the cell). In some embodiments, the verification code may be transmitted to the owner of the cell.

To generate or store reference values, a database of information related to cells may be maintained. For example, a database including information on cell lines, cell users, assay systems, or killing agents may be generated and maintained. The reference values may be stored in the database or generate at time-of-use from values in the database.

### IX. Example Kits

Embodiments may include a kit for verifying the destruction of a cell. The kit may include an agent configured to destroy the cell. The agent may be any agent described herein. The cell may be any cell described herein. In some embodiments, the kit may include a sample vial containing the cell.

The kit may also include a system for running an assay to detect a component in either the agent or a medium comprising a destroyed cell and the agent. The assay may be any assay described herein. The medium may be any medium described herein.

The system may be encoded with an indicator having a value. The value of the indicator may be generated after a medium is run through the assay using the system. The value of the indicator may depend on an identifier of the system, the vial, the agent. The system and the vial may each be labeled with an identifier. The agent may be in a container labeled with an identifier. The labels may or may not be visible to the user of the kit. The value may be any value described herein.

The kit may also include instructions stating that the user of the kit must provide evidence of cell destruction.

In yet further contemplated methods of verification, the inventor also contemplates system and methods in which it is ascertained that there are no longer living cells remaining using various methods, including optical, chemical, biochemical, and physical methods. For example, in one embodiment a standalone “box” and camera or video system could be employed that images the cells while the process is running. The so established system could then observe a state change of the cells, possibly by observing cells being lysed. Alternatively, or additionally, one or more optical systems already in use (e.g., for cell culture density determination) could be employed to monitor the entire cell culture throughout the cell production process, which can then witness the destruction process at the end of the growth. Suitable visual analysis could include size change, change in refractile appearance under phase contrast, color change in the presence of a vital dye, etc.

Another approach would be to have a protocol through which a user destroys the cells (e.g., using physical, chemical, mechanical, enzymatic, etc. methods) and then provides the destroyed cells to a verification party (which could be the same as the provider or a third party). The destroyed cells can then be analyzed to confirm identity and status. Advantageously, such method will also physically remove any cells from a user that may no longer have authorization to use the cells.

### X. Example Systems

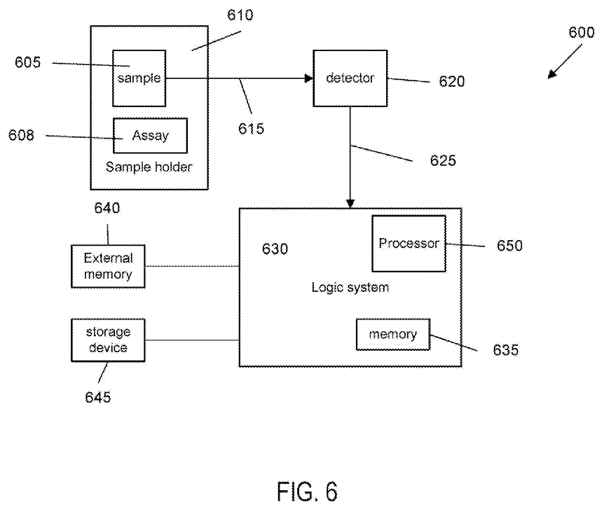

FIG. 6 illustrates a system 600 according to an embodiment of the present invention. The system as shown includes a sample 605, such as a cell medium containing a dead cell within a sample holder 610, where sample 605 can be contacted with an assay 608 to provide a signal of a physical characteristic 615. An example of a sample holder can be a flow cell that includes probes and/or primers of an assay or a tube through which a droplet moves (with the droplet including the assay). Physical characteristic 615 from the sample is detected by detector 620. Detector 620 can take a measurement at intervals (e.g., periodic intervals) to obtain data points that make up a data signal. In one embodiment, an analog to digital converter converts an analog signal from the detector into digital form at a plurality of times. Sample holder 610 and detector 620 can form a assay device. A data signal 625 is sent from detector 620 to logic system 630. Data signal 625 may be stored in a local memory 635, an external memory 640, or a storage device 645.

Logic system 630 may be, or may include, a computer system, ASIC, microprocessor, etc. It may also include or be coupled with a display (e.g., monitor, LED display, etc.) and a user input device (e.g., mouse, keyboard, buttons, etc.). Logic system 630 and the other components may be part of a stand-alone or network connected computer system, or they may be directly attached to or incorporated in a thermal cycler device. Logic system 630 may also include optimization software that executes in a processor 650. Logic system 630 may include a computer readable medium storing instructions for controlling system 600 to perform any of the methods described herein.

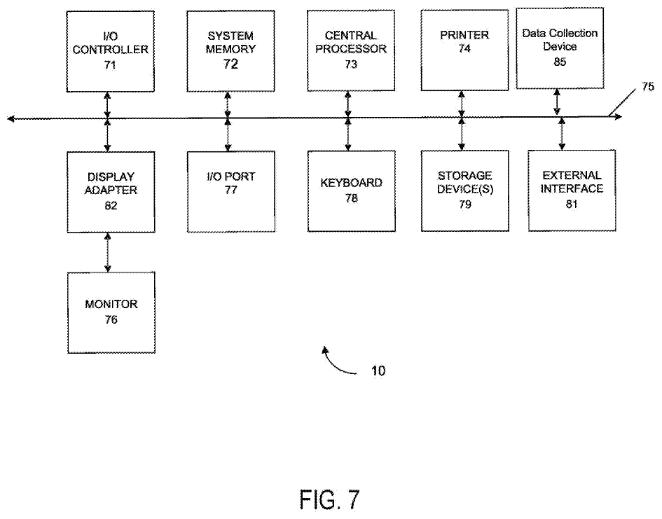

Any of the computer systems mentioned herein may utilize any suitable number of subsystems. Examples of such subsystems are shown in FIG. 7 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 7 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C #, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

In the preceding description, for the purposes of explanation, numerous details have been set forth in order to provide an understanding of various embodiments of the present technology. It will be apparent to one skilled in the art, however, that certain embodiments may be practiced without some of these details, or with additional details.

Having described several embodiments, it will be recognized by those of skill in the art that various modifications, alternative constructions, and equivalents may be used without departing from the spirit of the invention. Additionally, a number of well-known processes and elements have not been described in order to avoid unnecessarily obscuring the present invention. Additionally, details of any specific embodiment may not always be present in variations of that embodiment or may be added to other embodiments.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included.

As used herein and in the appended claims, the singular forms “a”, “an”, and “the” include plural referents unless the context clearly dictates otherwise. Thus, for example, reference to “a method” includes a plurality of such methods and reference to “the cell” includes reference to one or more cells and equivalents thereof known to those skilled in the art, and so forth. The invention has now been described in detail for the purposes of clarity and understanding. However, it will be appreciated that certain changes and modifications may be practice within the scope of the appended claims.

All publications, patents, and patent applications cited herein are hereby incorporated by reference in their entirety for all purposes. None is admitted to be prior art.

What is claimed is:

1. A method of verifying the destruction of a cell, the method comprising:
running a medium including at least a fragment of a dead cell through an assay using a system, wherein:
running the medium through the assay generates a value of an indicator to signify that the assay has been run;
transmitting the value to a processor; and
receiving a verification code from the processor to acknowledge that value is valid.

2. The method of claim 1, wherein the value identifies the system, the cell, or the destruction mode of the cell.

3. The method of claim 1, further comprising destroying the cell using an agent to produce the medium including at least the fragment of the dead cell.

4. The method of claim 1, wherein the destruction of the cell comprises the cell dying from apoptosis.

5. The method of claim 3, wherein the agent comprises a lysing agent, ultraviolet radiation, an antiseptic, a cytotoxin, a virus, a bacteria, a fungus, heat, cold, a cytolysis agent, an acid, a base, a white blood cell, or high pressure.

6. The method of claim 1, wherein the cell comprises an NK-92 cell or a T cell.

7. The method of claim 3, wherein:
the value identifies the destruction mode of the cell, and
the presence of the agent generates the value of the indicator.

8. The method of claim 1, wherein the indicator is a bar code, a QR code, an image, an alphanumeric string, a character string, a number, a line, or a color.

9. The method of claim 1, wherein:
the cell is a type of cell,
the value of the indicator comprises a portion associated with the type of cell, and
the value of the indicator depends on a proportion or an amount of a compound in the medium.

10. The method of claim 9, wherein the compound comprises perforin, granzymes, or a component specific to cell death.

11. The method of claim 9, wherein the value of the indicator is generated when the proportion or the amount of the compound exceeds a threshold.

12. The method of claim 1, wherein:
the value identifies the system,
the system is labeled with an identifier, and
the value depends on the identifier.

13. The method of claim 1, wherein:
the value identifies the cell,
the cell is tagged with a chemical or genomic moiety, and
the value depends on the chemical or genomic moiety.

14. The method of claim 1, wherein the verification code comprises a blockchain portion.

15. The method of claim 3, wherein:
the agent comprises a first reactant,
the assay comprises using a second reactant,
the method further comprising:
reacting the first reactant and the second reactant to produce a product, and
detecting the product in the medium to generate the value of the indicator to signify that the assay has been run.

16. The method of claim 1, wherein:
the assay is a lateral flow test,
the system comprises a lateral flow test substrate,
the indicator is a bar code or QR code,
the value comprises an identifier associated with the identity of the lateral flow substrate, and
the value of the indicator is generated by the value becoming visible after running the medium through the assay.

17. The method of claim 1, wherein:
the value identifies the destruction mode of the cell,
the medium comprises a plurality of dead cells and a plurality of live cells, and
the value depends on a proportion of dead cells and live cells.

18. The method of claim 1, wherein transmitting the value comprises sending an image of the value.

19. The method of claim 1, wherein:
the indicator is a light, and the value is a color; or
the indicator is an electrical or magnetic characteristic.

20. The method of claim 1, wherein:
the assay is a bioassay, a ligand binding assay, an immunoassay, an enzyme assay, a light detection system, a radioisotope assay, a polymerase chain reaction assay, a photometry assay, a transmittance assay, a turbidimetry assay, a nephelometry assay, a reflectometry assay, a viscoelastic measurement, a counting assay, an imaging assay, a cytometry assay, or an electrical detection.
